(19) United States
(12) Patent Application Publication
(10) Pub. No.: US 2011/0105604 A1
(43) Pub. Date: May 5, 2011

(54) MOISTURIZING AND CLEANSING COMPOSITION

(76) Inventors: Jason Sondgeroth, Ooltewah, TN (US); Elisa Barnes, Chattanooga, TN (US)

(21) Appl. No.: 12/940,183

(22) Filed: Nov. 5, 2010

Related U.S. Application Data
(60) Provisional application No. 61/258,295, filed on Nov. 5, 2009.

(51) Int. Cl. A61K 8/46; A61Q 19/00

(52) U.S. Cl. 514/517

(57) ABSTRACT

An enhanced cleansing and moisturizing composition that uses strong cationic surfactants and strong anionic surfactants to deposit more moisturizers onto a user's skin. The improved cleansing and moisturizing composition, generally includes a water-soluble strong cationic surfactant, a water-soluble strong anionic surfactant, a polymeric surfactant, and an aqueous carrier.

### CROSS-REFERENCE TO RELATED APPLICATIONS

This application is a non-provisional of and claims priority to U.S. Provisional Application No. 61/258,295 filed Nov. 5, 2009 which document is hereby incorporated by reference in its entirety to the extent permitted by law.

### BACKGROUND OF THE INVENTION

Dry skin is a common and often uncomfortable condition. Dry skin turns plump cells into shriveled ones and creates fine lines and wrinkles Skin can feel tight, painful, and itchy and look dull, red, and flakey. Intact skin is one of the body's primary defenses against infection. If skin is dried and cracked, additional avenues are created for bacteria to enter the body which can also lead to more serious health problems.

Normal healthy skin is coated in a thin layer of natural lipids or fatty substances. There are many different factors that lead to dry skin. Dry skin can appear anywhere but is most common on a person's hands, elbows, knees, lower legs and abdomen. It is often irritating and can be embarrassing. Often the environment or something a person does strips away these fatty oils, leaving skin unprotected. Other factors may include drying soaps, harsh soaps, itchy clothing, allergens, misusing moisturizers, frequent, hot showers, heavy exposure to sunlight, wind exposure, low-humid winters or other environmental conditions. Less often, the cause of dry skin is internal or genetic, although as hormone levels change, a person's skin also changes.

Cleansers or soaps that include moisturizers have been developed in response to the need for a cleanser that does not dry out the skin. Soap can quickly strip the skin's protective oils and dry out the skin. Therefore, it would be beneficial to provide a cleansing composition that also provides improved moisturization in order to improve poor or dry skin conditions.

Often these cleansers are in the form of a body wash or liquid product rather than a typical bar of soap. However, many of these cleansers are only slightly, if at all, more effective in fight dry skin and providing an increased amount of moisturizer to the skin than previous cleansers. These known cleanser formulas use weaker cationics that are designed to work in conjunction with anionics in order to deposit the moisturizers; however, weak cationics do not allow for a sufficient amount of moisturizer to be deposited on the skin. The stronger cationics deposit more moisturizer. Therefore, it would be beneficial to have a cleansing composition that used strong cationics and strong anionics to deposit a more effective amount of moisturizer on the skin. However, typically, combining strong cationics and strong anionics produces an insoluble precipitate that is formed due to the two charges combining and reacting. There is therefore a need in the art for a cleansing composition that uses strong cationics and strong anionics to deposit more moisturizers onto a user's skin where the strong cationics and strong anionics do not react to form a precipitate.

### SUMMARY OF THE INVENTION

The present invention provides an enhanced cleansing and moisturizing composition that uses strong cationic surfactants and strong anionic surfactants to deposit more moisturizers onto a user's skin. In one of many illustrative aspects of the present invention, there is provided an improved cleansing and moisturizing composition that includes a water-soluble strong cationic surfactant, a water-soluble strong anionic surfactant, a polymeric surfactant, and an aqueous carrier.

Other and further objects of the invention, together with the features of novelty appurtenant thereto, will appear in the course of the following description.

### DETAILED DESCRIPTION OF THE INVENTION

The present invention is generally directed to a cleansing and moisturizing composition. In particular, the composition of the present invention generally includes a water-soluble strong cationic surfactant, a water-soluble strong anionic surfactant, a water-soluble polymeric surfactant, and an aqueous carrier. The resulting composition provides a cleanser that deposits an effective amount of moisturizer onto the skin and combines strong cationic surfactants and strong anionic surfactants without the surfactants reacting and forming a precipitate.

The strong cationic surfactant of the composition of the present invention is generally present in an amount of from about 0.01% to about 10.0% by weight, more preferably from about 0.5% to about 3.0% by weight, and most preferably from about 1.0% to about 2.0% by weight of the composition. While not required, particularly preferred for use in the present invention are water-soluble, organic, cationic surfactants having a cationic charge density of greater than about 1.5 meq/gram and a molar mass of less than about 800 g/mol at the pH of the composition. Suitable cationic surfactants may include, but are not limited to, fatty quaternary amines such as alkyl ammonium chlorides, dimethyl dialkyl ammonium chlorides, alkyl ammonium methosulfates, alkyl ammonium chlorides, mixtures or combinations thereof. In certain embodiments, the cationic surfactant is behentrimonium methosulfate, distearyl dimonium chloride, mixtures or combinations thereof. In other embodiments, the cationic surfactant is an acid-neutralized amidoamine Suitable acid-neutralized amidoanines may include, but are not limited to, alkyl amdiopropyl dimethylanine, alkyl amidoethylethanolanine, iso-alkyl amidopropylmorpholine, alkyl amidopropylmorpholine, alkyl amidopropyldimethyl-amine, mixtures or combinations thereof.

The strong anionic surfactants of the composition of the present invention are generally present in an amount of from about 1.0% to about 50.0% by weight, more preferably from about 10.0% to about 30.0% by weight, and most preferably from about 15.0% to about 25.0% by weight of the composition. While not required, particularly preferred for use in the present invention are water-soluble, organic, anionic surfactants. Suitable anionic surfactants may include, but are not limited to, a primary alkane (C₈-C₂₂) sulfonate, a primary alkane (C₈-C₂₂) disulfonate, an alkane (C₈-C₂₂) sulfonate, a hydroxyalkane (C₈⁻C₂₂) sulfonate, an alkyl (C₁₂-C₁₈), or an alkyl ether sulfate, including alkyl glyceryl ether sulfates, where the cation is selected from one of sodium, potassium, ammonium or substituted ammonium, or combinations thereof. In certain embodiments, the anionic surfactant is ammonium lauryl ether sulfate, sodium lauryl ether sulfates, mixtures or combinations thereof. In other embodiments, the anionic surfactant is selected from one of the following: alkyl sulfosuccinates, including mono alkyl and dialkyl (C₆-C₂₂) sulfosuccinates; alkyl and acyl taurates; alkyl and acyl sarcosinates; sulfoacetates; alkyl (C₈-C₂₂) phosphates; phosphates; alkyl phosphate esters; alkoxyl alkyl phosphate esters; acyl lactates; monoalkyl (C₈⁻C₂₂) succinates; sulphoacetates; acyl isethionates; sarcosinates; taurates; lactylates; carboxylates; and mixtures or combinations thereof.

The polymeric surfactant of the composition of the present invention is generally present in an amount of from about 0.01% to about 10.0% by weight, more preferably from about 0.2% to about 0.6% by weight, and most preferably from about 0.3% to about 0.5% by weight of the composition. While not required, particularly preferred for use in the present invention are polymeric surfactants with a molar mass greater than 800 g/mol at the pH of the composition. In certain embodiments, the polymeric surfactant is PEG-30 diopolyhydroxystearate. In other embodiments, the polymeric surfactant has the general structure of PEG-(R)(M)-polyhydroxy-(S), wherein (R) is present in an amount of from about 1 mole to 50 moles, the (M) can be penta, tetra, tri, di or mono substituted, and the (S) can be a chain length from C₁₀ to C₃₀ and wherein the HLB value of the compound is less than eight.

In one embodiment of the present invention, the composition further includes one or more of a polyol, a skin conditioner, pH adjusters, mixtures, or combinations thereof. In another embodiment of the present invention, the composition may also further include silicone skin conditioning agents, emollients, mixtures, or combinations thereof.

Generally, the composition of the present invention has a viscosity of from about 10,000 to about 1,000,000 centipoise. In addition, water is generally present in the composition in an amount of from about 30.0% to 90.0% by weight, more preferably 30.0% to 70.0% by weight, and most preferably from about 40.0% to 50.0% by weight of the composition.

In one embodiment of the present invention, the composition may further include water-soluble vitamins such as, for example, vitamin B1, vitamin B2, vitamin B6, vitamin B12, vitamin C, pantothenic acid, pantothenyl ethyl ether, panthenol, biotin, derivatives and combinations thereof Another embodiment of the composition may further include water-soluble amino acids such as, for example, asparagine, alanin, indole, glutamic acid, salts and combinations thereof. Another embodiment of the composition may include water-insoluble vitamins such as, for example, vitamin A, vitamin D, vitamin E, derivatives and combinations thereof. A further embodiment of the composition may include water-insoluble amino acids such as, for example, tyrosine, tryptamine, salts and combinations thereof.

The composition of the present invention may also include other ingredients such as pigment materials including as, for example, inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, and combinations thereof. Other ingredients in the composition of the present invention may include chelating agents, fragrances, additives, benefit proteins, and combinations thereof.

In an illustrative but non-limiting example, an effective combination of the improved composition comprises about 44.0% by weight sodium laureth sulfate, about 22.0% by weight sodium lauroamphoacetate, about 12.0% by weight sodium methyl cocoyl taurate, about 7.50% by weight petrolatum, about 3.0% by weight glycerin, about 2.50% by weight hydrogenated vegetable oil, about 2.0% by weight lauric acid, about 2.0% by weight glyceryl stearate, about 1.54% by weight water, about 1.0% by weight cocamide MEA, about 1.0% by weight fragrance, about 0.84% by weight citric acid, about 0.50% by weight behentrimonium methosulfate and cetyl alcohol, about 0.40% by weight guar hydroxypropyltrimonium chloride, about 0.40% by weight polyquaternium-67, about 0.40% by weight PEG-30 dipolyhydroxystearate, about 0.20% by weight PEG-14M, about 0.20% by weight jojoba esters, about 0.10% by weight disodium EDTA, about 0.10% by weight shea butter, about 0.10% by weight aloe barbadensis leaf juice, about 0.10% by weight to copheryl acetate, about 0.10% by weight methylchloroisothiazolinone and methylisothiazolinone, about 0.01% by weight PEG-8 dimethicone and octyldodecanol and zea mays oil and tocopheryl acetate and ascorbic acid and phytonadione and cholecalciferol and silica and ethylhexyl palmitate and sodium propoxyhydroxypropyl thiosulfate silica, and about 0.01% by weight retinyl palmitate.

The compositions of the present invention, in general, may be made by mixing together water and any solids, such as waxes, that need to be melted at an elevated temperature. An appropriate elevated temperature is approximately 60° C. The ingredients are mixed thoroughly at the elevated temperature and then cooled to an ambient temperature. Any additional ingredients, including, but not limited to, electrolytes, fragrance, vitamins, botanicals, polymers, and particles, may then be added to the cooled product.

The body wash compositions of the present invention are used in a conventional manner for cleansing the skin. An effective amount of the composition for cleansing the skin is applied to the skin, lathered and then rinsed off. Such effective amounts generally range from about 0.2 gm to about 2 gm and most preferably from about 0.3 gm to about 0.5 gm.

This method for use on the skin comprises the steps of: a) applying an effective amount of the body wash to the skin, and b) lathering the hands or a device (e.g. wash cloth or poof), and c) rinsing. These steps can be repeated as many times as needed to achieve the desired cleansing and/or moisturization benefit.

From the foregoing it will be seen that this invention is one well adapted to attain all ends and objects hereinabove set forth together with the other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative, and notin a limiting sense.

What is claimed is:

1. A moisturizing and cleansing composition comprising:
from about 0.01% to about 10.0% by weight of a strong cationic surfactant having a cationic charge density of greater than about 1.6 meq/gram, wherein said cationic surfactant has a molar mass of less than about 800 g/mol at the pH of said composition;
from about 1.0% to about 50.0% by weight of a strong anionic surfactant;
from about 0.01% to about 10.0% by weight of a polymeric surfactant; and
an aqueous carrier.

2. The composition of claim 1 wherein said cationic surfactant is water-soluble and is a fatty quaternary amine.

3. The composition of claim 2 wherein said cationic surfactant is selected from the group consisting of alkyl ammonium chlorides, dimethyl dialkyl ammonium chlorides, alkyl ammonium methosulfates, alkyl ammonium chlorides, mixtures thereof, and combinations thereof.

4. The composition of claim 1 wherein said cationic surfactant is water-soluble and is selected from the group consisting of behentrimonium methosulfate, distearyl dimonium chloride, mixtures thereof, and combinations thereof.

5. The composition of claim 1 wherein said cationic surfactant is water-soluble and is selected from the group consisting of alkyl and amdiopropyl dimethylanine, alkyl amidoethylethanolanine, iso-alkyl amido propylmorpholine, alkyl amidopropylmorphine, alkyl amidopropyldimeththyl-anime, mixtures thereof, and combinations thereof.

6. The composition of claim 1 wherein said anionic surfactant is water-soluble and is selected from the group consisting of primary alkane sulfonates, primary alkanedisulfonates, alkene sulfonates, hydroxylalkane sulfonates, alkyl sulfates, alkyl ether sulfates, alkyl sulfosuccinates, alkyl taurates, acyl taurates, alkyl sarcosinates, acyl sarcosinates, sulfoacetates, alkyl phosphates, phosphates, alkyl phosphate esters, alkoxyl alkyl phosphate esters, acyl lactates, monoalkyl succinates, sulphoacetates, monoalkyl succinates, sulphoacetates, acyl isethionates, sarcosinates, taurates, lactylates, carboxylates, mixtures thereof, and combinations thereof.

7. The composition of claim 1 wherein said anionic surfactant is water-soluble and is selected from the group consisting of ammonium lauryl ether sulfate, sodium lauryl ether sulfate, mixtures thereof, and combinations thereof.

8. The composition of claim 1 wherein said polymeric surfactant has a molar mass greater than about 800 g/mol at the pH of said composition.

9. The composition of claim 8 wherein said polymeric surfactant is PEG-30 diopolyhydroxyetearate.

10. The composition of claim 8 wherein said polymeric surfactant has the general structure PEG-(R)(M)-polyhydroxy-(S), wherein said (R) ranges from about 1 to about 50 moles, said (M) is substituted with penta, tetra, tri, di, or mono, and said (S) is a carbon chain having from about 10 carbons to 30 carbons.

11. The composition of claim 10 wherein said polymeric surfactant has a HLB value of less than about 8.

12. The composition of claim 1 having a viscosity of from about 10,000 to about 1,000,000 centipoise.

13. The composition of claim 1 further comprising from about 1.0% to 90.0% by weight water.

14. The composition of claim 1, further comprising from about 0.0001% to about 99.0% by weight of a component selected from the group consisting of a polyol, a skin conditioner, a pH adjuster, achelating agent, fragrances, additives, benefit proteins, electrolytes, polymers, derivatives thereof, and combinations thereof.

15. The composition of claim 1, further comprising from about 0.0001% to about 99.0% by weight of a component selected from the group consisting of vitamins, amino acids, salts thereof, derivatives thereof, and combinations thereof.

16. The composition of claim 1, further comprising from about 0.0001% to about 99.0% by weight of a pigment material selected from the group consisting of inorganic, nitroso, monoazo, disazo, carotenoid, triphenyl methane, triaryl methane, xanthene, quinoline, oxazine, azine, anthraquinone, indigoid, thionindigold, quiacridone, phthalociannie, botanical and natural colors, derivatives thereof and combinations thereof.

17. A moisturizing and cleansing composition comprising:
from about 0.01% to about 10.0% by weight of a water-soluble, organic, strong cationic surfactant having a cationic charge density of greater than about 1.6 meq/gram, wherein said cationic surfactant has a molar mass of less than about 800 g/mol at the pH of said composition;
from about 1.0% to about 50.0% by weight of a water-soluble, organic, strong anionic surfactant;
from about 0.01% to about 10.0% by weight of a polymeric surfactant with a molar mass greater than about 800 g/mol at the pH of said composition; and
an aqueous carrier.

18. The composition of claim 17 wherein said cationic surfactant is from about 0.5% to about 3.0% by weight of said composition, said anionic surfactant is from about 10.0% to about 30.0% by weight of said composition, and said polymeric surfactant is from about 0.2% to about 0.6% by weight of said composition.

19. The composition of claim 17 wherein said cationic surfactant is from about 1.0% to about 2.0% by weight of said composition, said anionic surfactant is from about 15.0% to about 25.0% by weight of said composition, and said polymeric surfactant is from about 0.3% to about 0.5% by weight of said composition.

20. The composition of claim 19 wherein said cationic surfactant is selected from the group consisting of alkyl ammonium chlorides, dimethyl dialkyl ammonium chlorides, alkyl ammonium methosulfates, alkyl ammonium chlorides, mixtures thereof, and combinations thereof, behentrimonium methosulfate, distearyl dimonium chloride, mixtures thereof, or combinations thereof, alkyl and amdiopropyl dimethylanine, alkyl amidoethylethanolanine, iso-alkyl amido propylmorpholine, alkyl amidopropylmorphine, alkyl amidopropyldimeththyl-anime, mixtures thereof, and combinations thereof; wherein said anionic surfactant is selected from the group consisting of primary alkane sulfonates, primary alkanedisulfonates, alkene sulfonates, hydroxylalkane sulfonates, alkyl sulfates, alkyl ether sulfates, alkyl sulfosuccinates, alkyl taurates, acyl taurates, alkyl sarcosinates, acyl sarcosinates, sulfoacetates, alkyl phosphates, phosphates, alkyl phosphate esters, alkoxyl alkyl phosphate esters, acyl lactates, monoalkyl succinates, sulphoacetates, monoalkyl succinates, sulphoacetates, acyl isethionates, sarcosinates, taurates, lactylates, carboxylates, mixtures thereof, and combinations thereof; and wherein said polymeric surfactant has a general structure of PEG-(R)(M)-polyhydroxy-(S), wherein said (R) ranges from about 1 to about 50 moles, said (M) is substituted with penta, tetra, tri, di, or mono, and said (S) is a carbon chain having from about 10 carbons to 30 carbons and said composition further comprises from about 1.0% to about 90.0% by weight water.

21. A moisturizing and cleansing composition comprising:
about 44.0% by weight sodium laureth sulfate;
about 22.0% by weight sodium lauroamphoacetate;
about 12.0% by weight sodium methyl cocoyl taurate;
about 7.50% by weight petrolatum;
about 3.0% by weight glycerin;
about 2.50% by weight hydrogenated vegetable oil;
about 2.0% by weight lauric acid;
about 2.0% by weight glyceryl stearate;
about 1.0% by weight cocamide MEA;
about 1.0% by weight fragrance;
about 0.84% by weight citric acid;
about 0.50% by weight behentrimonium methosulfate and cetyl alcohol;
about 0.40% by weight guar hydroxypropyltrimonium chloride;
about 0.40% by weight polyquatemium-67;
about 0.40% by weight PEG-30 dipolyhydroxystearate;
about 0.20% by weight PEG-14M;
about 0.20% by weight jojoba esters;
about 0.10% by weight disodium EDTA;
about 0.10% by weight shea butter;
about 0.10% by weight aloe barbadensis leaf juice;
about 0.10% by weight to copheryl acetate;
about 0.10% by weight methylchloroisothiazolinone and methylisothiazolinone;
about 0.01% by weight PEG-8 dimethicone and octyldodecanol and zea mays oil and tocopheryl acetate and ascorbic acid and phytonadione and cholecalciferol and silica and ethyihexyl palmitate and sodium propoxyhydroxypropyl thiosulfate silica;
about 0.01% by weight retinyl palmitate; and
about 0.01% to 99.0% by weight water.
